(19) United States
(12) Patent Application Publication
(10) Pub. No.: US 2004/0127919 A1
(43) Pub. Date: Jul. 1, 2004

(54) Apparatus and method for reducing fluid loss during a surgical procedure

(76) Inventors: Hugh Trout III, Bethesda, MD (US); Frank Patterson, Exeter, NH (US)

Correspondence Address: COLLIER SHANNON SCOTT, PLLC, 3050 K STREET, NW, SUITE 400, WASHINGTON, DC, 20007, US

(21) Appl. No.: 10/667,521

(22) Filed: Sep. 23, 2003

Related U.S. Application Data
(60) Provisional application No. 60/412,549, filed on Sep. 23, 2002.
(60) Provisional application No. 60/415,778, filed on Oct. 4, 2002.

(51) Int. Cl. A61M 29/00

(52) U.S. Cl. 606/157; 606/200

(57) ABSTRACT

The present invention provides an occlusive system and method of use at a surgical procedure. The occlusive system comprises a fastener and an occlusive device in cooperation with the fastener. An embodiment of the invention may further comprise a fastener tip and a penetration apparatus. According to an embodiment of the present invention, a method of reducing the loss of fluid at a surgical site comprises the steps of: advancing an occlusive system comprising a fastener having an occlusive device to the surgical site; and deploying the surgical fastener having the occlusive device at the surgical site.

### CROSS-REFERENCE TO RELATED APPLICATIONS

The present invention relates to, and is entitled to the benefit of the earlier filing date and priority of, application Ser. No. 60/412,549, filed on Sep. 23, 2002, and application Ser. No. 60/415,778, filed on Oct. 4, 2002.

### FIELD OF THE INVENTION

The present invention relates to an apparatus and method for reducing fluid loss during a surgical procedure. In particular, the present invention relates to an apparatus and method for reducing blood loss from a vessel during a surgical procedure.

### BACKGROUND OF THE INVENTION

An aneurysm is a ballooning of the wall of an artery resulting from the weakening of the artery due to disease or other conditions. Left untreated, the aneurysm will frequently rupture, resulting in loss of blood through the rupture and death.

Aortic aneurysms are the most common form of arterial aneurysm and are life threatening. The aorta is the main artery which supplies blood to the circulatory system. The aorta arises from the left ventricle of the heart, passes upward and bends over behind the heart, and passes down through the thorax and abdomen. Among other arterial vessels branching off the aorta along its path, the abdominal aorta supplies two side vessels to the kidneys, the renal arteries. Below the level of the renal arteries, the abdominal aorta continues to about the level of the fourth lumbar vertebrae (or the navel), where it divides into the iliac arteries. The iliac arteries, in turn, supply blood to the lower extremities and perineal region.

It is common for an aortic aneurysm to occur in the portion of the abdominal aorta between the renal arteries and the iliac arteries. This portion of the abdominal aorta is particularly susceptible to weakening, often resulting in an aortic aneurysm. Such an aneurysm may be located near the iliac arteries. An aortic aneurysm larger than about 5 cm in diameter in this section of the aorta is ominous. Left untreated, the aneurysm may rupture, resulting in rapid, and usually fatal, hemorrhaging. Typically, a surgical procedure is not performed on aneurysms smaller than 5 cm because it has not been established that surgical intervention is beneficial for aneurysms that size.

Aneurysms in the abdominal aorta are associated with a particularly high mortality rate; accordingly, current medical standards call for urgent operative repair. Abdominal surgery, however, results in substantial stress to the body. Although the mortality rate for an aortic aneurysm is extremely high, there is also considerable mortality and morbidity associated with open surgical intervention to repair an aortic aneurysm. This intervention involves penetrating the abdominal wall to the location of the aneurysm to reinforce or replace the diseased section of the aortic aneurysm. A prosthetic device, typically a synthetic tube graft, is used for this purpose. The graft serves to exclude the aneurysm from the circulatory system, thus relieving pressure and stress on the weakened section of the aorta at the aneurysm.

Repair of an aortic aneurysm is a major operative procedure. Substantial morbidity accompanies the procedure, resulting in a protracted recovery period. Further, the procedure entails a substantial risk of mortality. While surgical intervention may be indicated, the surgery carries attendant risks and certain patients may not be able to tolerate the stress of intra-abdominal surgery. It is, therefore, desirable to reduce the mortality and morbidity associated with intra-abdominal surgical intervention.

In recent years, methods have been developed to attempt to treat an aortic aneurysm without the attendant risks of intra-abdominal surgical intervention. Among them are inventions disclosed and claimed in Kornberg, U.S. Pat. No. 4,562,596 for Aortic Graft, Device and Method for Performing an Intraluminal Abdominal Aortic Aneurysm Repair; Lazarus, U.S. Pat. No. 4,787,899 for Intraluminal Graft Device, System and Method; and Taheri, U.S. Pat. No. 5,042,707 for Intravascular Stapler, and Method of Operating Same.

None of the known systems provide an apparatus that selectively reduces blood loss from a vessel during attachment of a surgical component, such as a prosthetic graft, to a vessel wall in the manner of the embodiments of the present invention. In particular, attachment devices, including, but not limited to surgical fasteners, have been developed that are intended to attach a surgical component to tissue, component to component, or tissue to tissue. An example is the attachment of a prosthetic graft to an aortic wall as described in U.S. Pat. Nos. 5,944,750 and 5,957,940, which are hereby incorporated in their entirety by reference. When the attachment device is inserted independently, or in conjunction with a penetration apparatus, the attachment device will have an opening through its center that could allow the exodus of blood from within the aortic lumen unless some sort of occlusive mechanism is built into the attachment device itself or into an additional component used in conjunction with the attachment device.

Therefore, it is an advantage of some, but not necessarily all, embodiments of the present invention to provide a device that may be added either to the attachment device or to an additional component used in conjunction with the attachment device to reduce blood loss during a surgical procedure.

Additional advantages of various embodiments of the invention are set forth, in part, in the description that follows for reducing blood loss from a vessel during a surgical procedure.

### SUMMARY OF THE INVENTION

Responsive to the foregoing challenges, Applicant has developed an innovative apparatus and method for reducing blood loss from a vessel during a surgical procedure at a surgical site.

An alternative embodiment of the present invention is an occlusive system for use at a surgical site comprising a fastener and an occlusive device in cooperation with the fastener. The system may further comprise a fastener tip in communication with the fastener. The system may further comprise a penetration apparatus in communication with the fastener. The occlusive device may comprise one or more selected from the group consisting of a coil, band, ribbon, valve, and flap. In an alternative embodiment, the occlusive device may comprise at least one of a coil, band, ribbon, valve, flap, or any other suitable element.

In an alternative embodiment, the occlusive device may have a substantially closed configuration, or a first substantially open configuration and a second substantially closed configuration. In an alternative embodiment, the occlusive device may either collapse or expand into a substantially closed configuration. In an alternative embodiment, the occlusive device may be disposed within the fastener, adjacent to the fastener, or have a first portion disposed within the fastener and a second portion extending outside the fastener.

An alternative embodiment of the present invention is an occlusive system for use at a surgical site comprising a fastener, a fastener tip in cooperation with the fastener, and an occlusive device in cooperation with the fastener or the fastener tip. The occlusive device may be in cooperation with the fastener, the fastener tip, or both.

An alternative embodiment of the present invention is an occlusive system for use at a surgical site comprising a fastener, an occlusive device in cooperation with the fastener, and a penetration apparatus in reversible communication with the fastener and the occlusive device. The occlusive device may have a first substantially open configuration and a second substantially closed configuration.

An alternative embodiment of the present invention is an occlusive system for use at a surgical site comprising fastener means for fastening a first component to a second component, and occlusive means in cooperation with the fastener means for reducing fluid flow at the surgical site. The first component and second component may be comprised of a surgical component, a tissue, or both.

An alternative embodiment of the present invention is a method for reducing the loss of fluid at a surgical site comprising the steps of advancing an occlusive system comprising a fastener having an occlusive device to a surgical site and deploying the surgical fastener having the occlusive device at the surgical site. The method may further comprise the step of reducing the loss of fluid at the surgical site. The fluid may be blood, or any blood component.

It is to be understood that both the foregoing general description and the following detailed description are exemplary and explanatory only, and are not restrictive of the invention as claimed.

### BRIEF DESCRIPTION OF THE DRAWINGS

In order to assist the understanding of this invention, reference will now be made to the appended drawings, in which like reference characters refer to like elements.

FIG. 1A and B is a schematic view of an occlusive system in accordance with an embodiment of the present invention.

FIG. 2A is a cut-away pictorial view of an occlusive system including a penetration apparatus, fastener, occlusive device (not shown), and a fastener tip according to an embodiment of the present invention.

FIG. 2B is a perspective view of a fastener tip according to an embodiment of the present invention.

FIG. 3 is a cut-away pictorial view of the insertion of the penetration apparatus and the fastener through the surgical component and the vessel wall in accordance with an embodiment of the present invention.

FIG. 4 is a cut-away pictorial view of the fastener deployed through the surgical component and the vessel in accordance with an embodiment of the present invention.

FIGS. 5 and 6 are schematic views of an occlusive system according to an embodiment of the present invention.

FIG. 7A and B is a schematic view of an occlusive system according to an embodiment of the present invention.

FIG. 8A, B, C, D, E, and F is a perspective view of an occlusive device according to an embodiment of the present invention.

FIG. 9 is a cross-sectional view of an occlusive system according to an embodiment of the present invention having an opened flap valve.

FIG. 10 is a cross-sectional view of the embodiment of FIG. 9 with the flap valve in a closed configuration.

FIG. 11A and B is a schematic view of an example embodiment of the device comprising a thin wall material.

FIG. 12A and B is a schematic view of another example embodiment of the device comprising a thin wall material.

FIGS. 13, 14, and 15 are perspective views of further example embodiments of the device directed to a valve.

FIG. 16A and B is a schematic view of an embodiment of the device directed to the tapered tip.

FIG. 17A, B, and C is a schematic view of an embodiment of the device directed to a thin wall material.

FIG. 18A, B, and C is a schematic view of an example embodiment of the device directed to a polymeric cord.

FIGS. 19A and B and 20A, B, and C are schematic views of further embodiments of the occlusive device directed to an expandable material.

FIGS. 21 and 22 are cut-away pictorial views of further embodiments of the occlusive device directed to a mass of shape memory material in an opened and a closed configuration.

### DETAILED DESCRIPTION OF EMBODIMENTS OF THE INVENTION

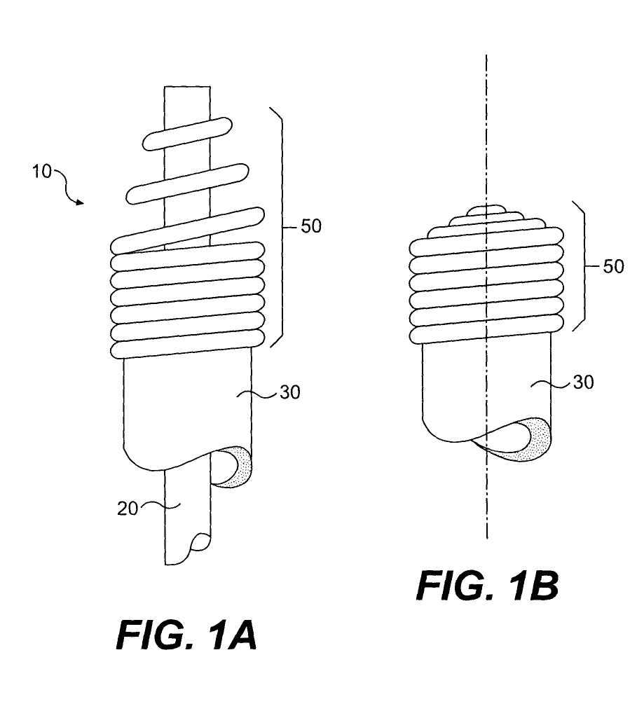

Reference will now be made in detail to various embodiments of the present invention, examples of which are illustrated in the accompanying drawings. As shown in FIG. 1, occlusive system 10 in accordance with an embodiment of the present invention comprises occlusive device 50 in communication with surgical fastener 30. Occlusive device 50 may be disposed within, or located adjacent to surgical fastener 30. Occlusive device 50 may comprise, but is not limited to, one or more coils, bands, ribbons, valves, flaps or any other suitable occlusive member. Occlusive device 50 may be static, expanding, and/or collapsing. In an alternative embodiment, occlusive device 50 may remain in a first substantially closed position, or may have a first substantially closed position and a second substantially open position. In an embodiment having a first and second position, occlusive device 50 is biased toward the first substantially closed position.

In alternative embodiments, occlusive system 10 comprises occlusive device 50 and surgical fastener 30 alone, or occlusive device 50 and surgical fastener 30 in combination with surgical fastener tip 40, and/or penetration apparatus 20, or any combination of the above. Various embodiments are discussed in greater detail in the following paragraphs.

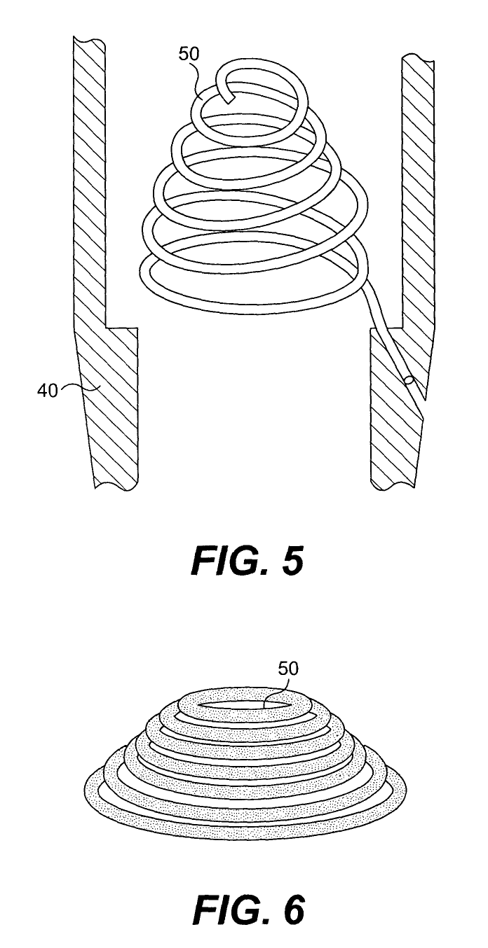

In an alternative embodiment, occlusive system 10 comprises occlusive device 50, surgical fastener 30, and surgical fastener tip 40. Surgical fastener tip 40 is located adjacent to surgical fastener 30. Occlusive device 50 may be disposed within and/or located adjacent to fastener 30, or disposed within and/or located adjacent to surgical fastener tip 40, as shown in FIG. 5.

In an alternative embodiment, occlusive system 10 may comprise occlusive device 50, surgical fastener 30, and penetration apparatus 20. Penetration apparatus 20 may comprise an optical fiber or any other apparatus to penetrate the surgical site including, but not limited to, a screw, a drill, a needle, or any other suitable mechanical or thermal energy means. Various embodiments of penetration apparatus 20 are described in U.S. Pat. Nos. 5,972,023; 5,957,940; 6,371,919; 5,997,556; 6,248,118; 6,520,974; 6,607,555, all of which are incorporated in their entirety by reference. When penetration apparatus 20 is used in conjunction with occlusive system 10, occlusive device 50 is maintained in the second substantially open position by the presence of penetration apparatus 20. Upon withdrawal of penetration apparatus 20 during the surgical procedure, occlusive device 50 assumes its first substantially closed position thereby reducing the loss of fluid at the surgical site. The fluid may be, but is not limited to, blood, blood products, and blood components. The surgical site may be within a lumen, such as, but not limited to a vessel.

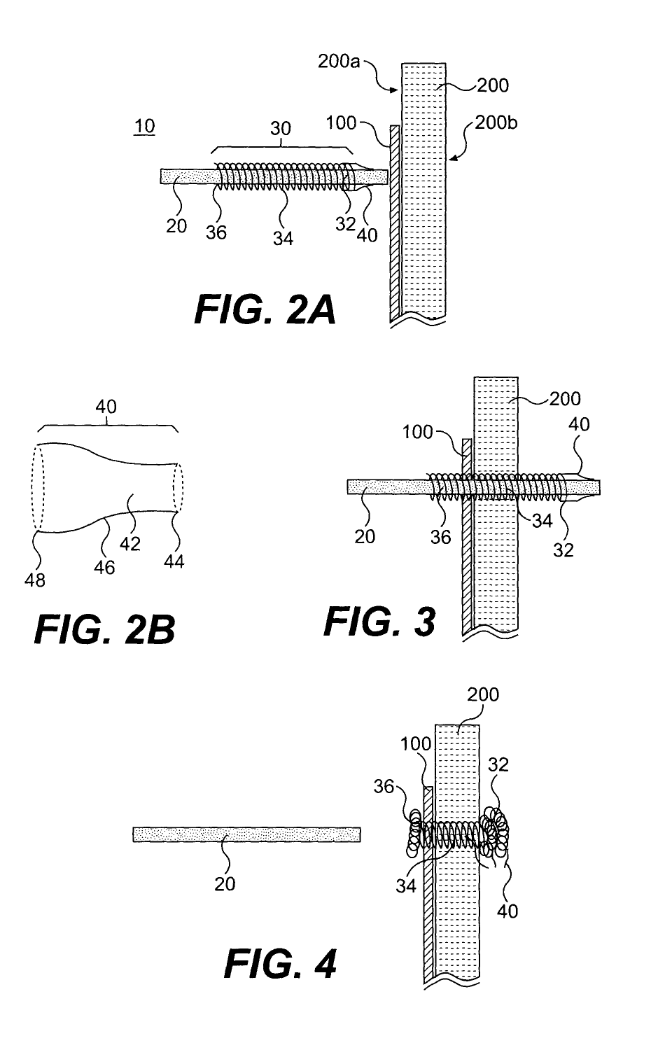

Occlusive system 10 in accordance with an alternative embodiment of the present invention is described in connection with FIGS. 2 through 4. In this embodiment, occlusive system 10 comprises fastener 30, occlusive device 50, tip 40, and penetration apparatus 20. Occlusive system 10 reduces blood loss when fastener 30 spans a surgical site. At the surgical site, occlusive system 10 advances through a lumen, such as, but not limited to, a vessel (not shown) that may have surgical component 100 adjacent to vessel wall 200, as shown in FIG. 2. In an example embodiment illustrated in FIG. 2, surgical component 100 and vessel wall 200 are shown with inside or interluminal potion 200A and outside or adventitial portion 200B. The surgical site, however, is not limited to a surgical component and a vessel but may be any combination of the surgical component and/or the vessel, such as, but not limited to, a surgical component to a surgical component, a surgical component to a vessel, or a vessel to a vessel.

In FIG. 2, fastener 30 is a flexible fastener that applies a force to attach surgical component 100 to the vessel wall 200, as disclosed in the following U.S. Patents and patent applications: U.S. Provisional Patent Application No. 60/181,230, filed Feb. 9, 2000; U.S. patent application Ser. No. 09/442,768, filed Nov. 18, 1999; U.S. patent application Ser. No. 09/213,233, filed Dec. 17, 1998, now U.S. Pat. No. 5,997,556; U.S. patent application Ser. No. 08/958,524, filed Oct. 27, 1997, now U.S. Pat. No. 5,957,940; U.S. patent application Ser. No. 08/896,415, filed Jul. 18, 1997, now U.S. Pat. No. 5,944,750; and U.S. Provisional Patent Application No. 60/051,209, filed Jun. 30, 1997. The subject matter of these patents and patent applications is incorporated herein in its entirety specifically by reference. In further example embodiments, fastener 30 may or may not apply a compressive force to attach surgical component 100 to vessel wall 200.

In an alternative embodiment shown in FIG. 2A, fastener 30 is disposed about or around penetration apparatus 20. Fastener 30 may be disposed about, around, proximal or distal to the surgical site, or within penetration apparatus 20. In further example embodiments, fastener 30 may be linked to penetration apparatus 20 in a manner such that the insertion of penetration apparatus 20 at the surgical site also includes the insertion of fastener 30, as illustrated in FIG. 3. Fastener 30 may be inserted at the surgical site before, concurrently with, or after, penetration apparatus 20. Fastener 30 may be sectioned into leading portion 32, middle portion 34, and trailing portion 36. Each portion of fastener 30 may be similar or different in structure and may be composed of similar or different materials such as, but not limited to, plastic, metal, metal alloy, or any other suitable material.

In the embodiment illustrated in FIG. 2A, fastener 30 is reversibly or irreversibly secured to tip 40. Tip 40 may facilitate penetration of the surgical site and in further embodiments, includes occlusive device 50 for reducing blood loss at the surgical site, as shown in FIG. 5. In one embodiment, tip 40 is attached to leading portion 32 of fastener 30, but also may be located elsewhere on fastener 30, such as, but not limited to, middle portion 34 and/or trailing portion 36. As shown in FIG. 2B, tip 40 may include inner lumen 42, leading edge 44, middle portion 46, and trailing edge 48. Tip 40 may facilitate penetration with or without the use of penetration apparatus 20, and may be composed of any suitable material, such as, but not limited to, plastic, metal, or metal alloy. Tip 40 shown in FIGS. 2A and 2B is of a conical or a bell shape, but may be of any suitable shape.

In further alternative embodiments of occlusive system 10, occlusive device 50 is housed within fastener 30 itself, located in one or more of leading portion 32, middle portion 34, or trailing portion 36, or into an apparatus attached to fastener 30, such as, but not limited to, tip 40. When penetration apparatus 20 is engaged with fastener 30 as depicted in FIG. 2A, occlusive device 50 assumes a second substantially open configuration. When penetration apparatus 20 is removed as shown in FIG. 4, occlusive device 50 assumes its first substantially closed configuration.

Various alternative embodiments of occlusive device 50 of occlusive system 10 will now be described with reference to FIGS. 1 and 6 through 22. In example embodiments illustrated in FIG. 1, occlusive device 50 comprises a spring winding or coil member that may be tapered and attached to fastener 30 and/or tip 40. Occlusive device 50 may be attached to fastener 30 in various ways such as, but not limited to, extension of the fastener wires, welded, threaded over the fastener, or any other suitable method of attachment. When penetration apparatus 20 is engaged with fastener 30 in FIG. 1A, occlusive device 50 is in a second substantially open configuration. When penetration apparatus 20 is removed, occlusive device 50 retracts toward fastener 30 into a first substantially closed configuration, as shown in FIG. 1 B. This retraction of occlusive device 50 assists in the reduction of fluid loss during and following the surgical procedure. Various embodiments of occlusive device 50 may be disposed at leading portion 32, middle portion 34, or trailing portion 36 of fastener 30 and/or with an apparatus, such as, but not limited to, tip 40. Illustrated in FIGS. 5 and 6, occlusive device 50 is located within tip 40.

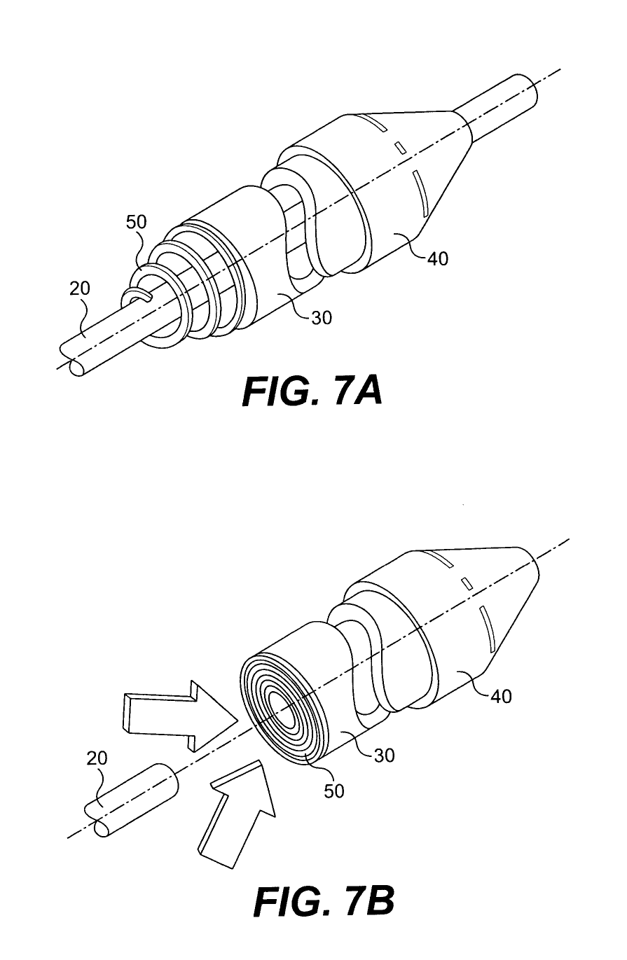

An alternative embodiment illustrated in FIGS. 7A and 7B shows occlusive device 50 disposed in trailing portion 36 of fastener 30. In particular, occlusive device 50 is comprised of a spring or spiral coil which may be fabricated from a solid disk. In FIGS. 7A and 7B, fastener 30 is also attached to tip 40 opposite occlusive device 50. When fastener 30 encompasses penetration apparatus 20, as in FIG. 7A, occlusive device 50 assumes a second substantially open configuration. When penetration apparatus 20 is withdrawn, as shown in FIG. 7B, occlusive device 50 assumes its first substantially closed configuration or retracted shape and provides resistance to fluid and/or blood flow. Occlusive device 50, according to this embodiment, may be fabricated from a metal, a polymeric material or any other suitable material and may be attached to fastener 30 in a number of ways, including, but not limited to, material specific welding, or any other suitable bonding techniques.

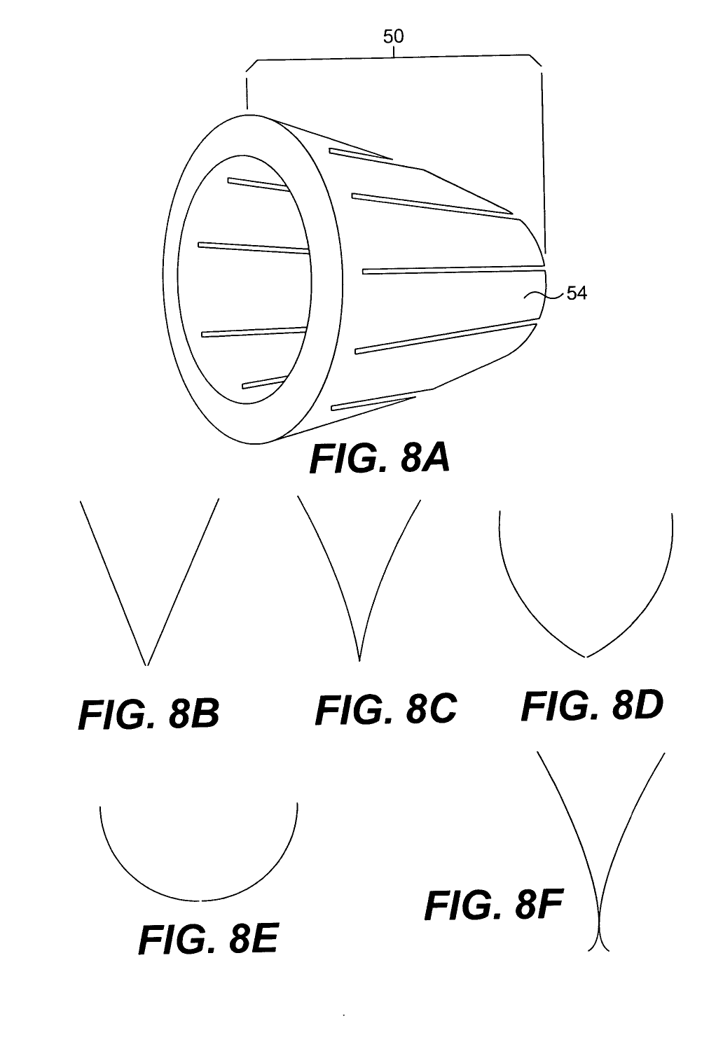

The alternative embodiments of occlusive device 50 shown in FIGS. 8A and 8B comprise a solid tapering cylinder with flap segments 52 that may be cut in a longitudinal manner and biased in a first substantially closed position. When penetration apparatus 20 is inserted through occlusive device 50, flap segments 52 may be distracted into a second substantially open position and when penetration apparatus 20 is withdrawn from occlusive device 50, flap segments 52 return to their first substantially closed position. In additional embodiments, occlusive device 50 is disposed within or attached to fastener 30 or into another structure such as, but not limited to, tip 40. FIGS. 8B through 8F depict further, non-limiting embodiments of occlusive device 50 with sagittal cuts there through. In particular, FIG. 8B is a straight cone; FIG. 8C is an insloping cone; FIG. 8D is an outsloping cone; FIG. 8E is a hemisphere; and FIG. 8F is an abbreviated hourglass.

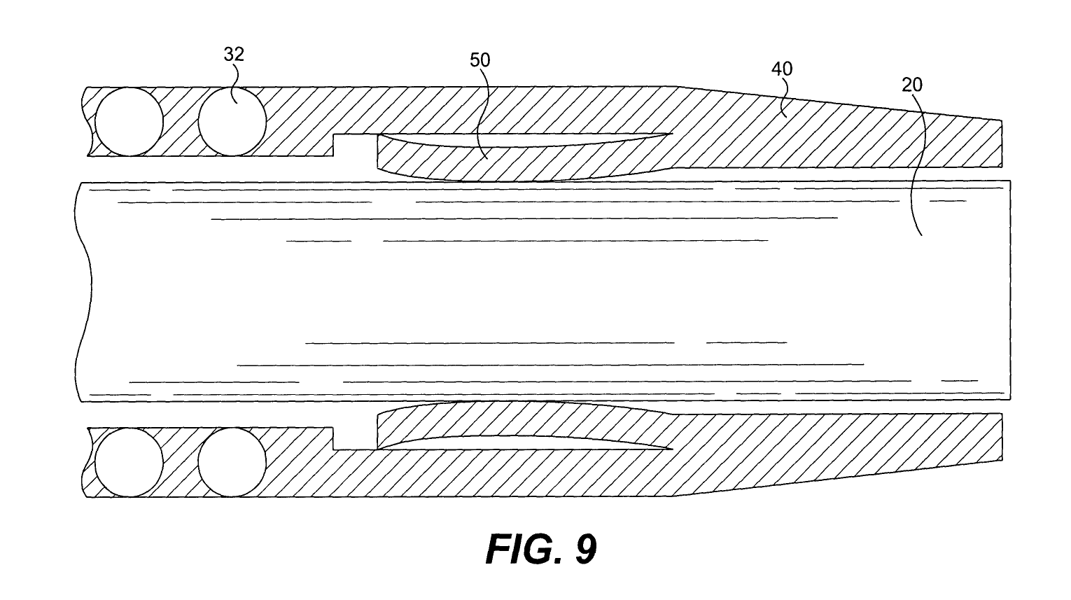

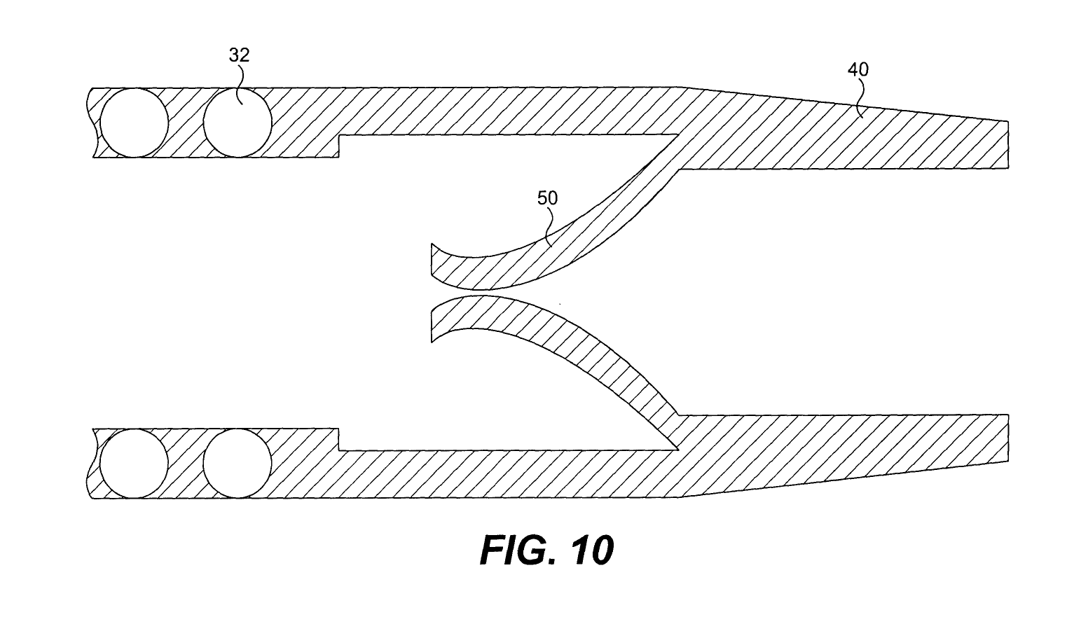

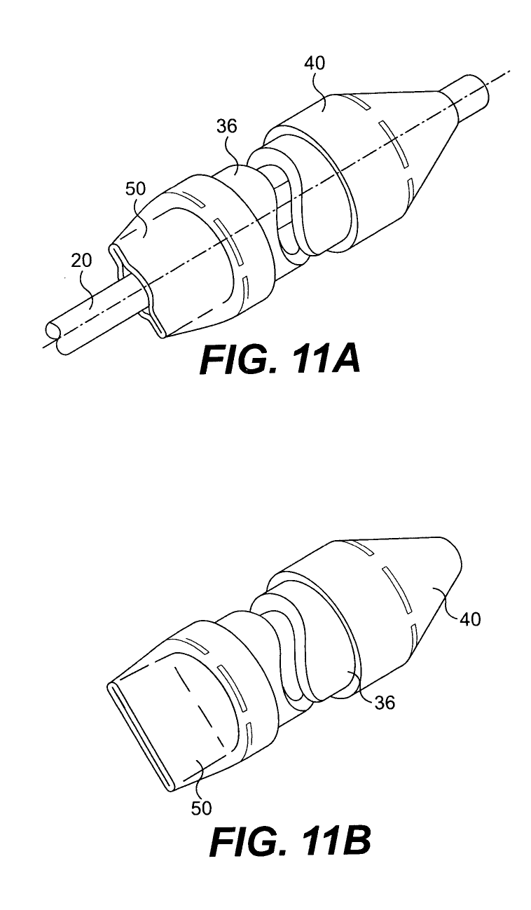

In the alternative embodiments of the present invention depicted in FIGS. 9 and 10, occlusive device 50 comprises a flap valve from a sagittal cut through tip 40 that may be attached to leading portion 32 of fastener 30. In FIG. 9, penetration apparatus 20 distracts occlusive device 50 such as those depicted in FIGS. 8A through 8F. In FIG. 10, penetration apparatus 20 is removed allowing occlusive device 50 to return to its first substantially closed position within tip 40.

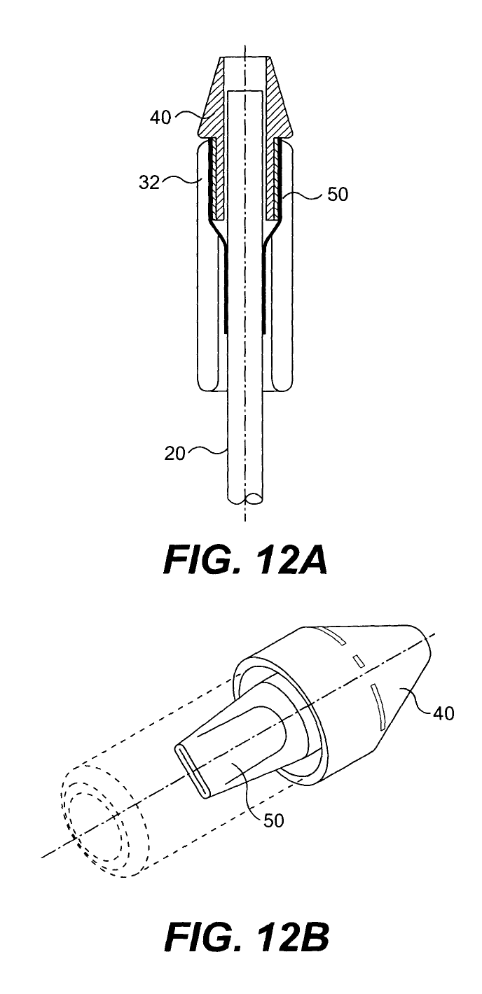

Another alternative embodiment of occlusive device 50 is shown in FIGS. 11 and 12. Occlusive device 50 comprises a thin wall material attached to tip 40, leading portion 32, middle portion 34, and/or trailing portion 36 of fastener 30 or at any other suitable location. The thin wall material of occlusive device 50 may comprise a thin wall tube, polymeric, biodegradable polymer or any other suitable material. In FIGS. 11A and 11B, occlusive device 50 may be preformed over trailing portion 36 of fastener 30. In FIG. 11A, a portion of penetration apparatus 20 is disposed within fastener 30 such that occlusive device 50 encircles penetration apparatus 20. In FIG. 11B, penetration apparatus 20 is withdrawn and occlusive device 50 collapses into its first substantially closed position assisting in the reduction of fluid and/or blood flow through fastener 30.

In a further alternative embodiment, occlusive device 50 illustrated in FIGS. 12A and 12B attaches to tip 40. The thin wall material comprising occlusive device 50 is preformed over trailing edge 48 of tip 40. Similarly, the thin wall material may be composed of thin wall tube, polymeric, biodegradable or any other suitable material. In FIG. 12A, penetration apparatus 20 extends through fastener 30 and is encircled by occlusive device 50. In FIG. 12B, the removal of penetration apparatus 20 collapses occlusive device 50 into its first substantially closed configuration resembling a “duck-bill” thereby reducing fluid and/or blood flow through fastener 30.

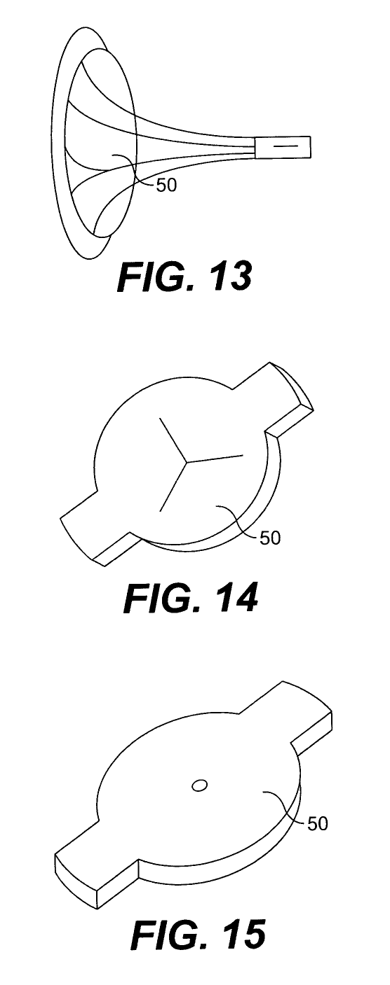

In FIGS. 13 through 15, additional alternative embodiments directed to occlusive device 50 are shown. In these figures, occlusive device 50 comprises a valve. Occlusive device 50 may be located within fastener 30 or within a separate apparatus to be attached to fastener 30, such as, but not limited to, tip 40. Occlusive device 50 may be constructed of metal alloys, synthetic material with or without elastomeric properties, or any other suitable materials. Occlusive device 50 of FIG. 13 is in a “duck-bill” configuration. In FIG. 14, occlusive device 50 is a tricuspid valve configuration having at least one cusp. Alternatively, in FIG. 15, occlusive device 50 is a radially expansive aperture valve. When penetration apparatus 20 inserts through occlusive device 50 illustrated in FIGS. 13 through 15, occlusive device 50 distracts or opens and when penetration apparatus 20 is removed, occlusive device 50 assumes its first substantially closed configuration. Occlusive device 50 depicted in FIGS. 13 through 15 is illustrated in the first substantially closed configuration.

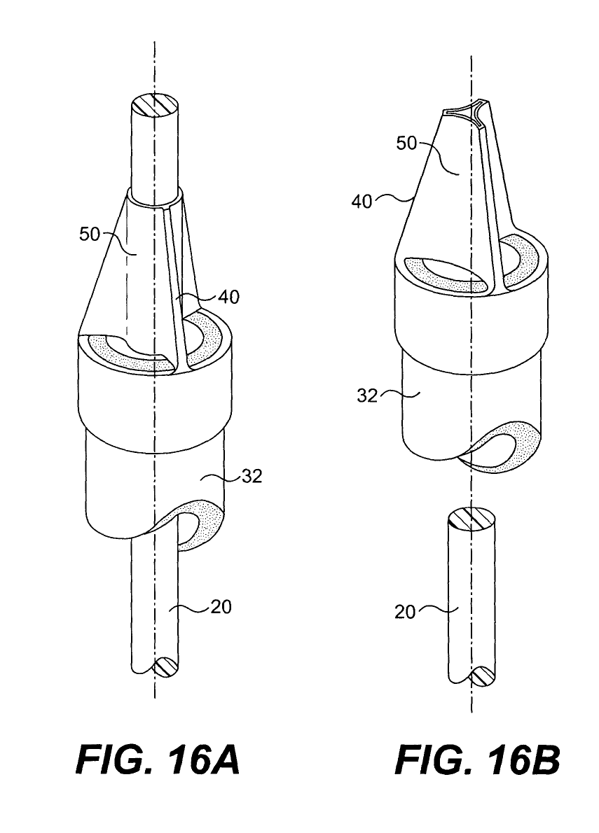

A further alternative embodiment of occlusive device 50 is illustrated in FIGS. 16A and 16B. Occlusive device 50 is integrated with tip 40 to form a unitary structure. In FIGS. 16A and 16B, occlusive device 50 comprises a circular cone having multiple webs that may be equispaced and/or identical. The embodiment of FIGS. 16A and 16B depicts tip 40 shaped in a cone having three webs. In alternative embodiments, tip 40 is threaded, crimped, shrunk or associated with leading portion 32 of fastener 30 or via any other suitable means. In FIG. 16A, penetration apparatus 20 is positioned within fastener 30 and distends occlusive device 50 of tip 40. As shown in FIG. 16B, penetration apparatus 20 is withdrawn resulting in a collapse of occlusive device 50 to its first substantially closed position to reduce fluid and/or blood flow through the fastener 30.

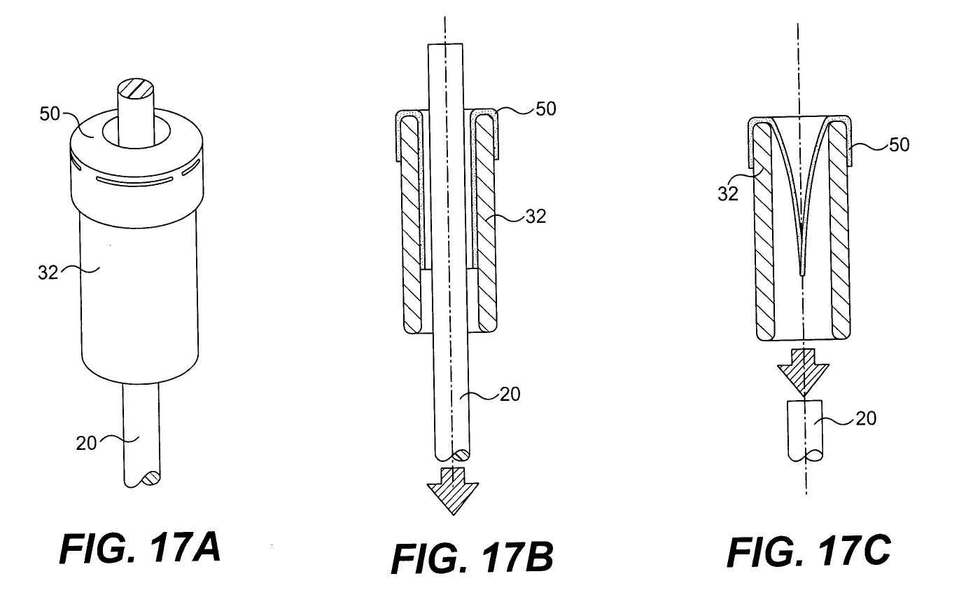

According to another alternative embodiment illustrated in FIGS. 17A and 17B, occlusive device 50 is comprised of a thin wall material that may be shrunk over leading portion 32 or trailing portion 36 of fastener 30. Occlusive device 50 may be secured to fastener 30 by crimping, screwing, shrink-wrapping, heating, welding, or attachment by any other suitable means. In example embodiments depicted in FIG. 17A, occlusive device 50 may be polymeric tubing shrunk over leading portion 32 of fastener 30. Illustrated in FIG. 17B, the cross section shows occlusive device 50 in a second substantially open configuration with penetration apparatus 20 in position within fastener 30. FIG. 17C depicts occlusive device 50 in its first substantially closed configuration after removal of penetration apparatus 20 from the surgical site.

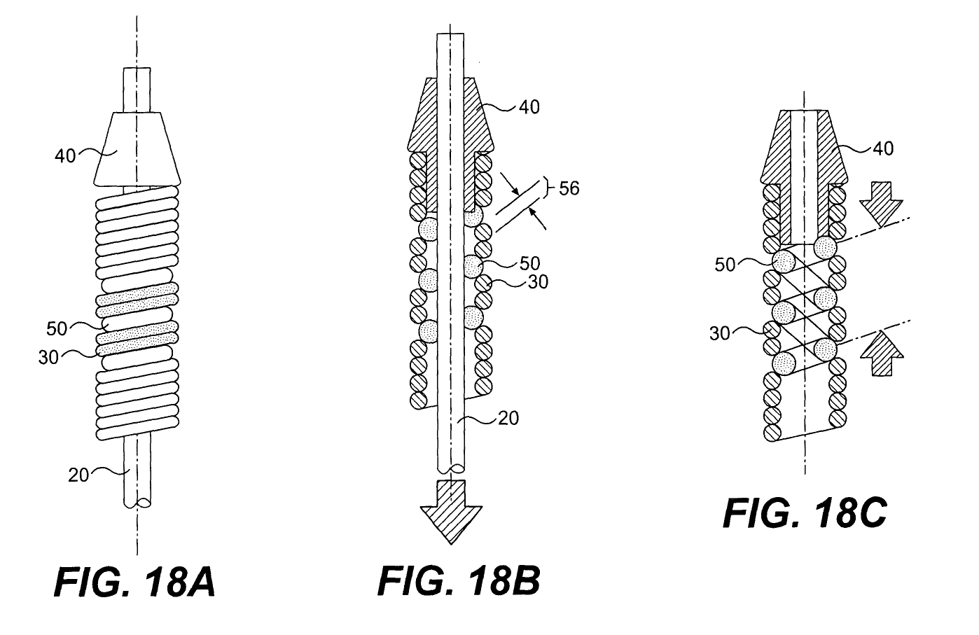

Further alternative embodiments of the invention are shown in FIGS. 18A and 18B. Occlusive device 50 may comprise a polymeric coil, ribbon, or cord positioned intermittently between and among a plurality of coils of fastener 30. Illustrated in FIG. 18A, occlusive device 50 is disposed in middle portion 34 of fastener 30 and around penetration apparatus 20, although occlusive device 50 may be disposed in leading portion 32 or trailing portion 36 of fastener 30. In FIG. 18B, separation 56 of occlusive device 50 is shown with fastener 30 and penetration apparatus 20. Occlusive device 50 is also in a second substantially open configuration in the presence of penetration apparatus 20. In contrast, FIG. 18C illustrates occlusive device 50 in its first substantially closed configuration with removal of penetration apparatus 20.

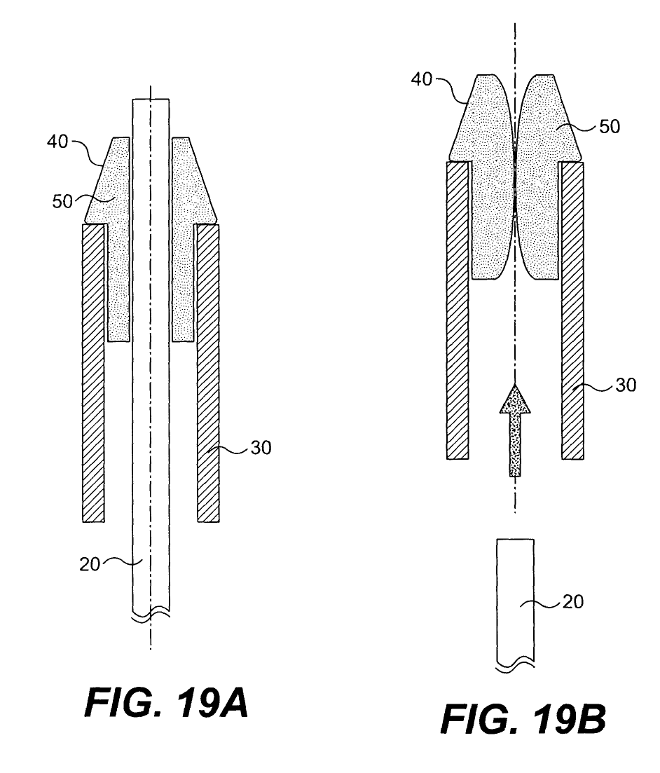

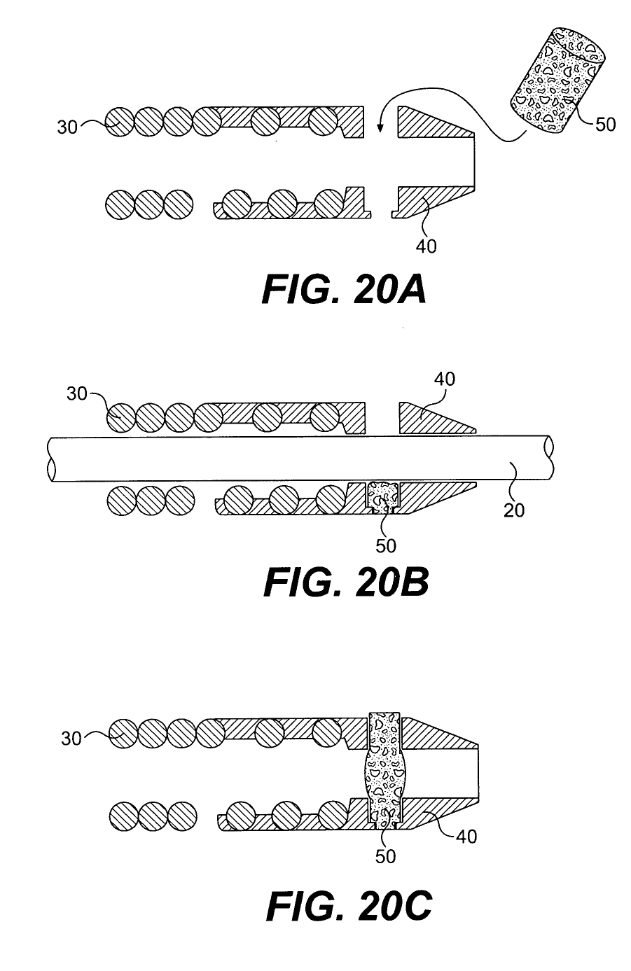

Various alternative embodiments of the present invention are also illustrated in FIGS. 19A and 19B. Occlusive device 50 may comprise one or more expandable members comprised of a polymeric foam or elastomer, or any other suitable expandable component. In an alternative embodiment, occlusive device 50 is located in tip 40 where it is compressed in the presence of penetration apparatus 20 into its second substantially open position, as shown in FIG. 19A. When penetration apparatus 20 is removed, as shown in FIG. 19B, occlusive device 50 may assume its first substantially closed position within tip 40 thereby reducing fluid and/or blood flow through fastener 30.

In other alternative embodiments, tip 40 may be composed of expandable material or biodegradable material (not shown). In this instance, fastener 30 is aligned over penetration apparatus 20 and over a reduced diameter of tip 40. With the removal of penetration apparatus 20, tip 40 may enlarge thereby forming a barrier to reduce the flow of fluid and/or blood through fastener 30.

As illustrated in FIGS. 20A, 20B and 20C, the expandable material of occlusive device 50 comprises an expandable plug. In various embodiments, occlusive device 50 is comprised of a polymeric or electrometric foam or any other suitable expandable component. Occlusive device 50, as illustrated in FIG. 20A and 20B, is positioned in a slot within tip 40. Tip 40 may be attached to fastener 30. With penetration apparatus 20 in place, as shown in FIG. 20B, occlusive device 50 is compressed and held in its second substantially open position. With the removal of penetration apparatus 20 from the surgical site, as depicted in FIG. 20C, occlusive device 50 may expand to its first substantially closed position, thereby assisting in the reduction of fluid and/or blood flow through fastener 30.

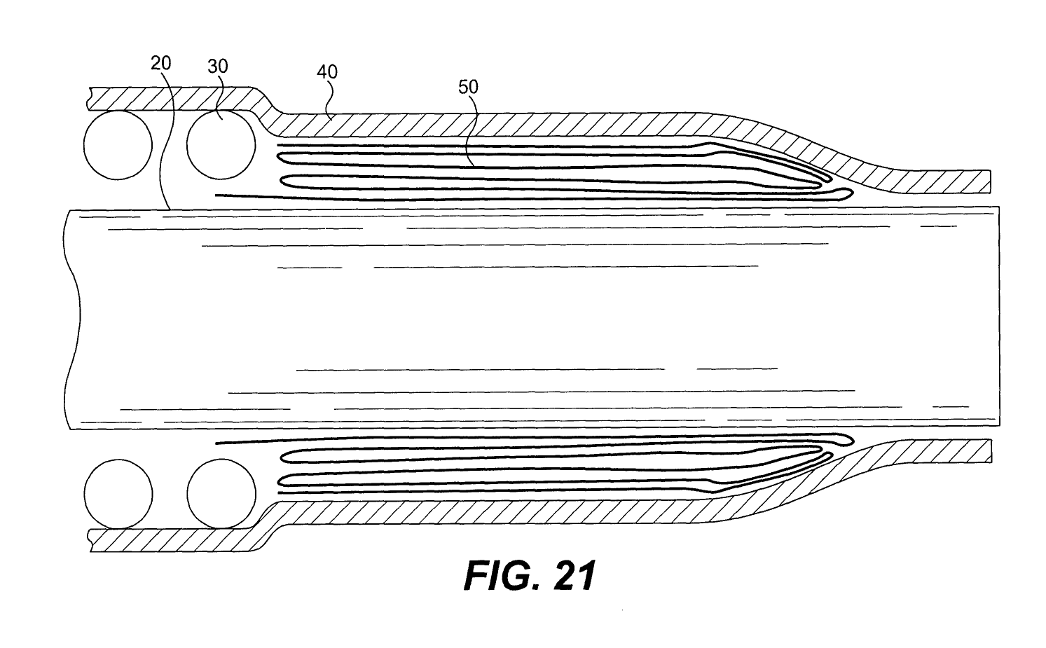

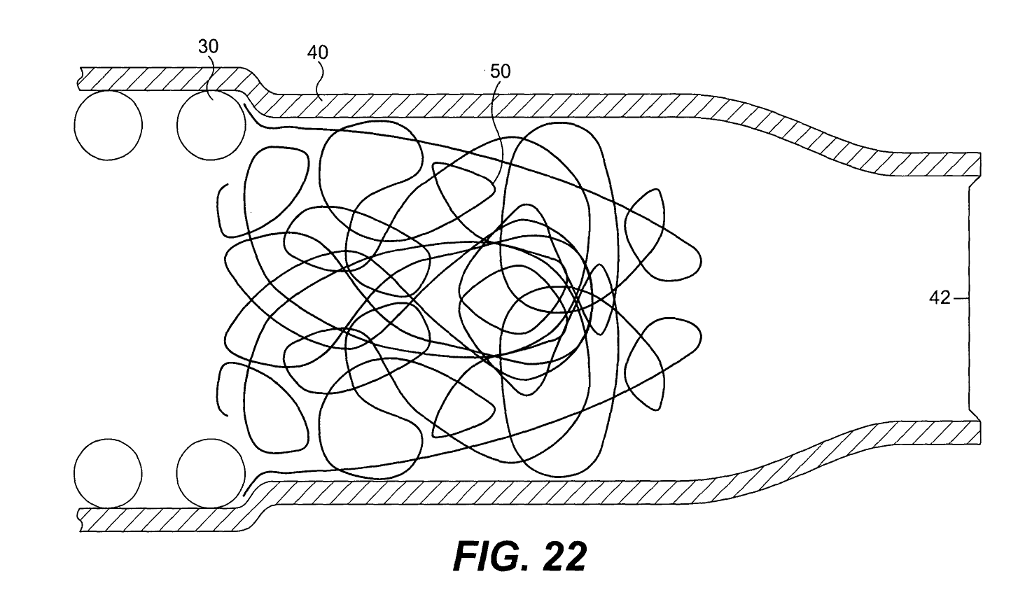

Example alternative embodiments illustrated in FIGS. 21 and 22 are directed to occlusive device 50 comprising a mass of shaped memory material. Occlusive device 50 may be comprised of coils, ribbon, and/or wire or any other suitable material disposed within tip 40 or any other appropriate location within tip 40 or fastener 30. In FIG. 21, occlusive device 50 is in a deformed second substantially open configuration allowing penetration apparatus 20 to pass through fastener 30. In FIG. 22 with the removal of penetration apparatus 20 from the surgical site, occlusive device 50 assumes its first substantially closed or pre-formed configuration thereby inhibiting the flow of fluid and/or blood through fastener 30. Occlusive device 50 may also possess thrombic properties to inhibit the blood flow. Occlusive device 50 may assume a configuration of such a size that it would not be able to move through fastener 30 or tip 40.

An embodiment of the method of operating occlusive system 10 will now be described. Occlusive system 10 comprising fastener 30 and occlusive device 50 is advanced to a surgical site through a lumen, such as, but not limited to, a vessel as disclosed in the following U.S. Patents and patent applications: U.S. Provisional Patent Application No. 60/181,230, filed Feb. 9, 2000; U.S. patent application Ser. No. 09/442,768, filed Nov. 18, 1999; U.S. patent application Ser. No. 09/213,233, filed Dec. 17, 1998, now U.S. Pat. No. 5,997,556; U.S. patent application Ser. No. 08/958,524, filed Oct. 27, 1997, now U.S. Pat. No. 5,957,940; U.S. patent application Ser. No. 08/896,415, filed Jul. 18, 1997, now U.S. Pat. No. 5,944,750; and U.S. Provisional Patent Application No. 60/051,209, filed Jun. 30, 1997, incorporated in their entirety specifically by reference. Occlusive system 10 is deployed at the surgical site attaching surgical component 100 to vessel wall 200, or surgical component to surgical component, or vessel to vessel, or any combination of the above. Occlusive device 50 in its first substantially closed position reduces blood loss through fastener 30 during and following the surgical procedure.

In an alternative embodiment shown in FIG. 2A, occlusive system 10 is advanced to the surgical site through a lumen, such as, but not limited to, a vessel (not shown). Penetration apparatus 20 is advanced either simultaneously or in sequence with tip 40 of fastener 30 to contact surgical component 100. Penetration apparatus 20 positioned with fastener 30 engages occlusive device 50 resulting in a second substantially open configuration. Penetration apparatus 20 and/or tip 40 creates an aperture within surgical component 100 and vessel wall 200. As indicated in FIG. 3, penetration apparatus 20 extends through surgical component 100 and vessel wall 200 such that fastener 30 also extends through surgical component 100 and vessel wall 200. In FIG. 4, penetration apparatus 20 is withdrawn from within fastener 30. Leading portion 32 of fastener 30 attached to tip 40 and trailing portion 36 resume their preformed coiled coil shape while middle portion 34 extends through surgical component 100 and vessel wall 200. With the withdrawal of penetration apparatus 20, occlusive device 50 assumes its first substantially closed configuration thereby reducing the flow of blood from adventitial portion 200B to luminal portion 200A and/or vice versa (not shown). Occlusive system 10 may be deployed at the surgical site as described in U.S. Pat. Nos. 5,972,023; 5,957,940; 6,371,919; 5,997,556; 6,248,118; 6,520,974; 6,607,555, and U.S. patent application Ser. No. 10/417,163, filed Apr. 17, 2003, herein incorporated in their entirety by reference. Occlusive system 10 may be advanced to the surgical site and deployed without the use of a penetration apparatus 20, or without the use of a surgical tip 40. Various embodiments of occlusive system 10 may be used to attach one or more of a surgical component, such as, but not limited to a prosthetic graft, and a tissue, such as, but not limited to a vessel. In one example embodiment occlusive system 10 comprising occlusive device 50 and surgical fastener 30 is deployed during the surgical procedure to attach a surgical component to a vessel, a surgical component to another surgical component, a vessel to vessel, or any combination thereof.

Numerous characteristics and advantages have been set forth in the foregoing description, together with details of structure and function. The novel features are pointed out in the appended claims. The disclosure, however, is illustrative only, and changes may be made in detail, especially in matters of shape, size and arrangement of parts, within the principle of the invention, to the full extent indicated by the broad general meaning of the terms in which the appended claims are expressed.

What is claimed is:

1. An occlusive system for use at a surgical site comprising:
a fastener; and
an occlusive device in cooperation with the fastener.

2. The system of claim 1, further comprising a fastener tip in communication with the fastener.

3. The system of claim 1, further comprising a penetration apparatus in communication with the fastener.

4. The system of claim 1, wherein the occlusive device comprises one or more selected from the group consisting of a coil, band, ribbon, valve, and flap.

5. The system of claim 1, wherein the occlusive device comprises at least one coil.

6. The system of claim 1, wherein the occlusive device comprises at least one band.

7. The system of claim 1, wherein the occlusive device comprises at least one ribbon.

8. The system of claim 1, wherein the occlusive device comprises at least one valve.

9. The system of claim 1, wherein the occlusive device comprises at least one flap.

10. The system of claim 1, the occlusive device having a substantially closed configuration.

11. The system of claim 1, the occlusive device having a first substantially open configuration and a second substantially closed configuration.

12. The system of claim 1, wherein the occlusive device collapses into a substantially closed configuration.

13. The system of claim 1, wherein the occlusive device expands into a substantially closed configuration.

14. The system of claim 1, wherein the occlusive device is disposed within the fastener.

15. The system of claim 1, wherein the occlusive device is disposed adjacent to the fastener.

16. An occlusive system for use at a surgical site comprising:
a fastener;
a fastener tip in cooperation with the fastener; and
an occlusive device in cooperation with the fastener or the fastener tip.

17. The system of claim 16, wherein the occlusive device is in cooperation with the fastener.

18. The system of claim 16, wherein the occlusive device is in cooperation with the fastener tip.

19. An occlusive system for use at a surgical site comprising:
a fastener;
an occlusive device in cooperation with the fastener; and
a penetration apparatus in reversible communication with the fastener and the occlusive device.

20. The system of claim 19, the occlusive device having a first substantially open configuration and a second substantially closed configuration.

21. An occlusive system for use at a surgical site comprising:
fastener means for fastening a first component to a second component; and
occlusive means in cooperation with the fastener means for reducing fluid flow at the surgical site.

22. A method for reducing the loss of fluid at a surgical site comprising the steps of:
advancing an occlusive system comprising a fastener having an occlusive device to a surgical site; and
deploying the surgical fastener having the occlusive device at the surgical site.

23. The method of claim 22, further comprising the step of:
reducing the loss of fluid at the surgical site.

24. The method of claim 22, wherein the fluid is blood.
